(19) United States
(12) Patent Application Publication
(10) Pub. No.: US 2009/0220614 A1
(43) Pub. Date: Sep. 3, 2009

(54) Thermo-Responsive Block Co-Polymers, and Use Thereof

(75) Inventors: Shuhui Qin, Barlett, IL (US); Shu Yang, Blue Bell, PA (US); Dennis Discher, Philadelphia, PA (US); Yan Geng, Langhorne, PA (US)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: The Trustees of the University of Pennsylvania

(21) Appl. No.: 12/086,680

(22) PCT Filed: Dec. 15, 2006
(86) PCT No.: PCT/US06/48103

§ 371 Date: Jan. 22, 2009

Related U.S. Application Data
(60) Provisional application No. 60/751,521, filed on Dec. 19, 2005.

(51) Int. Cl. A61K 9/14

(52) U.S. Cl. 424/501

(57) ABSTRACT

Provided are thermo-responsive polymersomes, which display cold-controlled encapsulation near the physiological temperatures, and have a PDI less than 1.2. Morphology of the thermo-responsive polymersomes is a function of the weight fraction of the hydrophilic block in the block copolymer and the number average molecular weight (Mₙ) of the block copolymer. When the lower critical solution temperature (LCST) is at, or slightly above physiological temperature, the thermo-responsive block displays hydrophobic properties, such that the block copolymer self-assembles in aqueous solution to form a polymersome with the thermo-responsive block occupying the core of the polymersome and the hydrophilic block occupying the corona of the polymersome. Below the LCST, the thermo-responsive block displays hydrophilic properties, such that the polymersome dissociates, providing fast release of an active agent encapsulated therein.

### FIELD OF THE INVENTION

The present invention is related to the field of block copolymers. More specifically, the present invention is related to block copolymers that display either hydrophilic or amphiphilic properties in response to external stimuli.

### BACKGROUND OF THE INVENTION

Vesicles and biomembranes from natural amphiphiles, phospholipids, play important roles in nutrient transport, DNA protection and specific targeting in cell functions. Compared to small spherical micelles (tens of nanometers in diameter), microscopic vesicles can entrap larger-sized molecules, including hydrophobic compositions within the core, and hydrophilic ones within the membrane shell, respectively. Therefore, developing vesicle-forming materials has attracted much interest for the applications ranging from cosmetics or nutrients to drug delivery.

Synthetic vesicles assembled from amphiphilic block copolymers, polymersomes, offer several material design and performance advantages over vesicles from small molecular weight surfactants and biological lipids. The rational design and synthesis of well-defined block copolymers, having desired molecular weight, volume fraction, and chemistry, have been shown to improve the vesicle stability while retaining the fluidity and deformability similar to that of lipid vesicles. In particular, poly(ethylene oxide) (PEO) based polymersomes have been demonstrated as robust drug delivery vehicles for controlled encapsulation, transportation, and release of the encapsulated material. PEO, notable for its biocompatibility and resistance to protein adsorption and cellular adhesion, has been used as the hydrophilic block to slow down the reticuloendothelial system (RES) clearance, resulting in prolonged circulation time in vivo (Ma et al., Biomacromol. 4(4):864-868 (2003)). It will be highly desirable to design “stimuli-responsive” PEO-based vesicles, that is ones that entrap soluble substance in water, and maintain their stability during the circulation, but become effectively destabilized upon a specific environmental stimulus to fast release the encapsulants when reaching the target.

Responsive block copolymer self-assemblies that are sensitive to external stimuli, including temperature, pH, electrolyte concentration and electrical potentials are of great interest as novel containers, micro-reactors and actuators to mimic natural systems. Some well-defined thermo-responsive block copolymers have been produced by incorporating thermo-responsive polymers into hydrophilic polymers by group transfer polymerization and living radical polymerization. Thermo-responsive polymers, such as poly[2-(diethylamino)ethyl methacrylate], poly[2-(diisopropylamino)ethyl methacrylate], poly[2-(N-morpholino)ethyl methacrylate] and poly(N-isopropylacrylamide) (“PNIPAAm”) exhibit reversible phase transition from hydrophilic to hydrophobic, and vice versa, in aqueous solution at a lower critical solution temperature (LCST). In particular, poly(ethylene oxide)-block-poly(N-isopropylacrylamide) (“PEO-b-PNIPAAm”) has been synthesized as a thermoresponsive gels and surface modifier (Yoshinari et al., Polymer 2005, 46, 7741 (2005); Iwai et al., Lumin. 2000, 87:1289 (2000); Kaholek et al., Chem. Mater. 16:3688 (2004); Kaholek et al., Nano Lett. 4:373 (2004)), but little is known of its ability to self-assemble in water. Spherical micelles or gels—but not micelles—have been observed above the LCST over a surprisingly wide range of f_{PEO} values (0.25-53.8 wt %) (Zhang et al., Macromol. 38:5743 (2005); Zhu et al., Langmuir 16:8543 (2000); Zhu et al., Macromol. 32:2068 (1999); Topp et al., Macromol. 30:8518 (1997); Motokawa et al., Macromol. 38:5748 (2005)). Moreover, with the exception of Zhang et al., Biomacromol. 38:5743 (2005), that reported spherical micelles from narrow-distribution PEO-b-PNIPAAm (PDI=1.06, ƒ_{PEO}=50 wt %), synthesized by atom transfer radical polymerization (ATRP), such diblocks have been synthesized by conventional free-radical polymerization methods, including Ce(IV) ion redox reaction and 2,2′-azobis(2-methylpropionitrile) (AIBN) type poly(ethylene glycol) (PEG) macroinitiator polymerization. Unfortunately, however, these methods typically result in a broad polydispersity index (PDI>1.5).

Accordingly, until the present invention, there remained a need in the art for a thermo-responsive block copolymer that is capable of polymersome formation in a useful temperature range, i.e., near physiological temperatures.

### SUMMARY OF THE INVENTION

The present invention provides for the first time, thermo-responsive giant micelles, polymersomes, which self-assemble from well-defined block copolymers of PEO-b-PNIPAAm, with a PDI less than 1.2. The thermo-responsive polymersomes display cold-controlled encapsulation and fast release near the physiological temperature. By varying functionality, composition and molecular weight, the morphologies of the molecular assemblies and their reaction to stimuli can be controlled. Thus, it is an object of the invention to provide a polymersome comprising a thermo-responsive block copolymer that is capable of polymersome formation. One such block copolymer comprises a hydrophilic block comprising polyethylene glycol terminated with an alkyl ether, and a thermo-responsive block comprising a poly N-alkylacrylamide, poly N-alkylaminoacrylate, or copolymer thereof.

When the LCST is at, or slightly above physiological temperature, the thermo-responsive block displays hydrophobic properties, such that the block copolymer self-assembles in aqueous solution to form a polymersome with the thermo-responsive block occupying the core of the polymersome and the hydrophilic block occupying the corona of the polymersome. Below the LCST, the thermo-responsive block displays hydrophilic properties, such that the polymersome dissociates. The morphology of the polymersome that is formed according to the current invention is a function of the weight fraction of the hydrophilic block in the block copolymer and the number average molecular weight (Mₙ) of the block copolymer.

It is also an object of the invention to provide a method for encapsulating a hydrophobic molecule, e.g., an active agent. The present invention also provides thermo-responsive polymersomes which encapsulate one or more “active agents,” which include, without limitation compositions such as a drug, therapeutic compound, dye, nutrient, sugar, vitamin, protein or protein fragment, salt, electrolyte, gene or gene fragment, product of genetic engineering, steroid, adjuvant, biosealant, gas, ferrofluid, or liquid crystal. The thus “loaded” polymersome may be further used to transport an encapsulatable material (an “encapsulant”) to or from its immediately surrounding environment.

Moreover, the present invention provides methods of using the thermo-responsive polymersome or encapsulating membrane to transport one or more of the above identified compositions to or from a patient in need of such transport activity. For example, the polymersome could be used to deliver a drug or therapeutic composition to a patient's tissue or blood stream, or it could be used to remove a toxic composition from the blood stream of a patient with, for example, a life threatening hormone or enzyme imbalance. Also provided by the present invention are methods of preparing an “empty” polymersome, wherein the preferred methods of preparation include at least one step consisting of a film rehydrating step, a bulk rehydrating step, or an electroforming step.

Further provided are methods for controlling the release of an encapsulated material from a thermo-responsive polymersome by modulating and controlling the composition of the membrane, specifically by providing a thermo-responsive block copolymer according to the invention and forming an aqueous solution or suspension of the block copolymer and a hydrophobic molecule to be encapsulated therein. The aqueous solution or suspension is heated to a temperature at, or above, the LCST, wherein the LCST is itself at, or above, physiological temperature, thus triggering self-assembly of the block copolymer into a polymersome and encapsulating the hydrophobic molecule in the core of the polymersome.

It is further an object of the invention to provide a method for delivering an active agent, e.g., drug or other hydrophobic species. The method comprises providing a block copolymer according to the current invention and forming an aqueous solution or suspension of the selected block copolymer and an active agent or other species to be delivered. Prepared as above, the block copolymers self-assemble into a plurality of thermo-responsive polymersomes, thereby encapsulating the active agent or other species into the core of the polymersomes. In general, following delivery of the polymersomes to a target, the target area is locally cooled to a temperature below the LCST, thereby causing the controlled dissociation of the polymersomes and release of the encapsulated active agent. The process according to this embodiment of the present invention can be practiced for the delivery of active agents, including drugs and other compositions, to either living patients or for in vitro studies.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 shows GPC traces of PEO macromolecular agent (solid) and poly(ethylene oxide-b-N-isopropyl-acrylamide (PEO-b-PNIPAAm) (dashed line).

FIG. 2 shows a series of images of the temperature-dependent self-assembly of PEO-b-PNIPAAm in aqueous solution.

FIGS. 3A-3C show images of the selected morphologies of PEO-b-PNIPAAm copolymer in aqueous solution above LCST. FIG. 3A shows highly ordered vesicles (Mₙ=25,000; ƒ_{EO}=8%). FIG. 3B shows branched worms (Mₙ=5,500; ƒ_{EO}=36%). FIG. 3C shows short rods (Mₙ=3,500; ƒ_{EO}=58%).

FIGS. 4A and 4B show the ¹H NMR spectra of the dithiobenzol-end-capped PEO macro chain transfer agent (FIG. 4A) and PEO-b-PNIPAAm (FIG. 4B).

FIG. 5 shows GPC traces of PEO macro chain transfer agent (solid) and PEO-b-PNIPAAm copolymer, OPA₂₁₅ (dashed line) PEO Mₙ=2000, PDI=1.09; OPA₂₁₅ PEO Mₙ=26,300, PDI=1.09.

FIGS. 6A and 6B show the temperature-dependent assemblies of OPA₂₁₅ vesicles (0.25 mg ml⁻¹). FIG. 6A shows the mean Dₕ measured by dynamic light scattering (DLS). Inset: size distribution of vesicles formed at 37° C. FIG. 6B shows optical transmission at 500 nm measured by UV-vis spectrometry. Lines are provided to guide the eye.

FIGS. 7A-7B show the conformation of the temperature-responsive polymersomes in a membrane stability study of OPA₂₁₅ vesicles encapsulating sucrose (320 mOsm) suspended in isotonic (FIG. 7A) and 400 mOsm PBS (FIG. 7B) solutions.

FIG. 8 shows the morphologic effect of cold-controlled release of sucrose encapsulated in an OPA₂₁₅ vesicle.

FIG. 9 shows release of doxorubicin from OPA₂₁₅ vesicles at 37° C. and 27° C. Lines are provided to guide the eye.

### DETAILED DESCRIPTION OF CERTAIN EMBODIMENTS OF THE INVENTION

The formation of “polymersomes,” stable vesicles comprising large semi-permeable, thin-walled encapsulating membranes, self-assembled in aqueous solutions of amphiphilic block copolymers, has been previously disclosed in published U.S. Patent Applications US2005/0003016, US2005/0048110, US2005/0180922, and U.S. Pat. No. 6,835,394, all to Discher et al, the contents of which are hereby incorporated by reference in their entirety. The “thermo-responsive” block copolymers according to the current invention are, however, unique in that they transition from hydrophilic to amphiphilic in response to changes in solution temperature. The embodied thermo-responsive block copolymers comprise: 1) a hydrophilic block, which is an alkyl ether terminated polyethylene glycol (polyethylene oxide); and 2) a thermo-responsive block, which comprises an N-alkylacrylamide, N-alkylaminoacrylate or a copolymer thereof. The thermo-responsive block copolymers according to the current invention are preferably produced via reversible addition-fragmentation chain transfer (RAFT) polymerization. RAFT polymerization is an important technique for living/controlled radical polymerization of a wide range of monomers, including nitrogen atom containing monomers. Recently PEO-b-PNIPAAm and PNIPAAm-b-PEO-b-PNIPAAm copolymers have been described using PEO capped with one or two dithiobenzoyl groups as macro chain transfer agents (Hong et al., supra 2004).

A relevant class of super-amphiphilic molecules is represented by block copolymers, e.g., hydrophilic polyethyleneoxide (PEO) linked to hydrophobic polyethylethylene (PEE). The synthetic diversity of block copolymers provides the opportunity to make a wide variety of vesicles with material properties that greatly expand what is currently available from the spectrum of naturally occurring phospholipids. In a preferred embodiment, the invention further provides for the preparation of vesicles harboring mixtures of super-amphiphiles and smaller amphiphiles, such as phospholipids up to at least 20% mole fraction. The latter have been shown to be capable of integrating into stable vesicles of super-amphiphiles.

“Vesicles,” as the term is used in the present invention, are essentially semi-permeable bags of aqueous solution as surrounded (without edges) by a self-assembled, stable membrane composed predominantly, by mass, of either amphiphiles or super-amphiphiles which self-assemble in water or aqueous solution. Thus, a biological cell would, in general, represent a naturally occurring vesicle. Smaller vesicles are also found within biological cells, and many of the structures within a cell are vesicular. The membrane of an internal vesicle serves the same purpose as the plasma membrane, i.e., to maintain a difference in composition and an osmotic balance between the interior of the vesicle and the exterior. Many additional functions of cell membranes, such as in providing a two-dimensional scaffold for energy conversion can be added to compartmentalization roles. For an intracellular vesicle, the environment outside the vesicle is the cytoplasm.

The “cell membrane” or “plasma membrane” is a complex, contiguous, self-assembled, complex fluid structure comprised of amphiphilic lipids in a bilayer with associated proteins and which defines the boundary of every cell. It is also referred to as a “biomembrane.” “Phospholipids” comprise lipid substances, which occur in cellular membranes and contain esters of phosphoric acid, such as sphingomyelins, and include phosphatides, phospholipins and phospholipoids.

Synthetic amphiphiles having molecular weights in the range of a few kilodaltons, like natural amphiphiles, are pervasive as self-assembled, encapsulating membranes in water-based systems. These include complex fluids, soaps, lubricants, microemulsions consisting of oil droplets in water, as well as biomedical devices such as vesicles. An “encapsulating membrane,” as the term is used in the present invention, is a vesicle in all respects except for the necessity of aqueous solution. Encapsulating membranes, by definition, compartmentalize by being semi- or selectively permeable to solutes, either contained inside or maintained outside of the spatial volume delimited by the membrane. Thus, a vesicle is a capsule in aqueous solution, which also contains aqueous solution. However, the interior or exterior of the capsule could also be another fluid, such as an oil or a gas. A “capsule,” as the term is used in the present invention, is the encapsulating membrane plus the space enclosed within the membrane.

“Complex fluids” are fluids that are made from molecules that interact and self-associate, conferring novel rheological, optical, or mechanical properties on the fluid itself. Complex fluids are found throughout biological and chemical systems, and include materials such as biological membranes or biomembranes, polymer melts and blends, and liquid crystals. The self-association and ordering of the molecules within the fluid depends on the interaction between component parts of the molecules, relative to their interaction with solvent, if present.

The plasma membrane is a “lipid bilayer” comprising a double layer of phospholipid/diacyl chains, wherein the hydrophobic fatty acid tails of the phospholipids face each other and the hydrophilic polar heads of each layer face outward toward the aqueous solutions. Numerous receptors, steroids, transporters and the like are embedded within the bilayer of a typical cell. Thus, a “lipid vesicle” or “liposome,” is a vesicle surrounded by a membrane comprising one or more phospholipids. Throughout the specification the terms “cell membrane,” “plasma membrane,” “lipid membrane,” and “biomembrane” may be used interchangeably to refer to the same lipid bilayer surrounding a cell or vesicle.

A “membrane,” as the term is used in this invention, is a spatially distinct collection of molecules that defines a 2-dimensional surface in 3-dimensional space, and thus separates one space from another in at least a local sense. Such a membrane must also be semi-permeable to solutes. It must also be sub-microscopic (less than optical wavelengths of around 500 nm) in its thickness, as resulting from a process of self-assembly. It can have fluid or solid properties, depending on temperature and on the chemistry of the amphiphiles from which it is formed. At some temperatures, the membrane can be fluid (having a measurable viscosity), or it can be solid-like, with an elasticity and bending rigidity. The membrane can store energy through its mechanical deformation, or it can store electrical energy by maintaining a transmembrane potential. Under some conditions, membranes can adhere to each other and coalesce (fuse). Soluble amphiphiles can bind to, and intercalate within a membrane.

A “bilayer membrane” (or simply “bilayer(s)”) for the purposes of this invention is a self assembled membrane of amphiphiles or super-amphiphiles in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self assembly,” a spontaneous, entropy-driven process of preparing a closed semi-permeable membrane.

Because of the bilayer's perselectivity, materials may be “encapsulated” in the aqueous interior or intercalated into the hydrophobic membrane core of the polymersome vesicle of the present invention. Numerous technologies can be developed from such vesicles, owing to the numerous unique features of the bilayer membrane, particularly with the addition of the thermally controlled dissociation of the present invention, and the broad availability of block copolymer amphiphiles.

The synthetic thermo-responsive polymersome membrane can exchange material with the “bulk,” i.e., the solution surrounding the vesicles. Each component in the bulk has a partition coefficient, meaning it has a certain probability of staying in the bulk, as well as a probability of remaining in the membrane. Conditions can be predetermined so that the partition coefficient of a selected type of molecule will be much higher within a vesicle's membrane, thereby permitting the polymersome to decrease the concentration of a molecule, such as cholesterol, in the surrounding bulk, permitting encapsulation of a selected molecule. Conversely, the monomeric units may be of a single type (homogeneous), or a variety of types (controlling the partition coefficient, the molecule is released into the bulk when the environment surrounding the polymersome has a higher partition coefficient.

The polymersomes of the present invention are formed from synthetic, amphiphilic copolymers. An “amphiphilic” substance is one containing both polar (water-soluble) and hydrophobic (water-insoluble) groups. “Polymers” are macromolecules comprising connected monomeric heterogeneous molecules. The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths which, when covalently attached to a phospholipid, optimize the circulation life of a liposome, is known to be in the approximate range of 34-114 covalently linked monomers (EO34 to EO114).

The preferred class of polymer selected to prepare the polymersomes of the present invention is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, χ, which provides a measure of the energetic cost of placing a monomer of A next to a monomer of B. Generally, the segregation of polymers into different ordered structures in the melt is controlled by the magnitude of χN, where N is proportional to molecular weight. For example, the tendency to form lamellar phases with block copolymers in the melt increases as χN increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material. In the melt, numerous structural phases have been seen for simple AB diblock copolymers.

To form a stable membrane in water, the absolute minimum requisite molecular weight for an amphiphile must exceed that of methanol HOCH₃, which is undoubtedly the smallest canonical amphiphile, with one end polar (HO⁻) and the other end hydrophobic (⁻CH₃). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile (see, Israelachvili, in Intermolecular and Surface Forces, 2nd ed., Pt3 (Academic Press, New York) 1995).

The most common lamellae-forming amphiphiles also have a hydrophilic volume fraction between 20 and 50%. Such molecules form, in aqueous solutions, bilayer membranes with hydrophobic cores never more than a few nanometers in thickness. The ability of amphiphilic and super-amphiphilic molecules to self-assemble can be largely assessed, without undue experimentation, by suspending the synthetic super-amphiphile in aqueous solution and looking for lamellar and vesicular structures as judged by simple observation under any basic optical microscope or through the scattering of light.

For typical phospholipids with two acyl chains, temperature can affect the stability of the thin lamellar structures, in part, by determining the volume of the hydrophobic portion. In addition, the strength of the hydrophobic interaction, which drives self-assembly and is required to maintain membrane stability, is generally recognized as rapidly decreasing for temperatures above approximately 50° C. Such vesicles generally are not able to retain their contents for any significant length of time under conditions of boiling water.

Upper limits on the molecular weight of synthetic amphiphiles which form single component, encapsulating membranes clearly exceed the many kilodalton range, as concluded from the work of Discher et al., Science 284:1143 (1999), which contributes foundationally to the present invention, and is herein incorporated by reference.

Block copolymers with molecular weights ranging from about 2 to 10 kilograms per mole can be synthesized and made into vesicles when the hydrophobic volume fraction is between about 20% and 50%. Diblocks containing polybutadiene are prepared, for example, from the polymerization of butadiene in cyclohexane at 40° C. using sec-butyllithium as the initiator. Microstructure can be adjusted through the use of various polar modifiers. For example, pure cyclohexane yields 93% 1, 4 and 7% 1, 2 addition, while the addition of THF (50 parts per Li) leads to 90% 1, 2 repeat units. The reaction may be terminated with, for example, ethyleneoxide, which does not propagate with a lithium counterion and HCl, leading to a monofunctional alcohol. This PB-OH intermediate, when hydrogenated over a palladium (Pd) support catalyst, produces PEE-OH. Reduction of this species with potassium naphthalide, followed by the subsequent addition of a measured quantity of ethylene oxide, results in the PEO-PEE diblock copolymer. Many variations on this method, as well as alternative methods of synthesis of diblock copolymers are known in the art; however, this particular preferred method is provided by example, and one of ordinary skill in the art would be able to prepare the selected diblock copolymer.

In yet another example, triblock copolymers having a PEO end group can also form polymersomes using similar techniques. Various combinations are possible comprising, e.g., polyethylene, polyethylethylene, polystyrene, polybutadiene, and the like. For example, a polystyrene (PS)—PB-PEO polymer can be prepared by the sequential addition of styrene and butadiene in cyclohexane with hydroxyl functionalization, re-initiation and polymerization. PB-PEE-PEO results from the two-step polymerization of butadiene, first in cyclohexane, then in the presence of tetrahydrofuran (THF), hydrolyl functionalization, selective catalytic hydrogenation of the 1, 2PB units, and the addition of the PEO block. ABC triblocks can range from molecular weights of 3,000 to at least 30,000 g/mol. Hydrophilic compositions should range from 20-50% in volume fraction, which will favor vesicle formation. The molecular weights must be high enough to ensure hydrophobic block segregation to the membrane core. The Flory interaction parameter between water and the chosen hydrophobic block should be high enough to ensure segregation. Symmetry can range from symmetric ABC triblock copolymers (where A and C are of the same molecular weight) to highly asymmetric triblock copolymers (where, for example, the C block is small, and the A and B blocks are of equal length)

### A. Preparation of Thermo-Responsive Polymersomes

In the preferred embodiments of the present invention, the thermo-responsive polymersomes transition from hydrophilic to amphiphilic in response to changes in solution temperature. Vesicle formation and block copolymer synthesis are two different things. The vesicles are prepared by dissolving the synthesized block copolymers in water or an aqueous co-solvent. The block copolymers according to the current invention are preferably synthesized by anionic polymerization, atom transfer radical polymerization (ATRP), nitroxide mediated polymerization (NMP), group transfer polymerization (GTP) and reversible addition-fragmentation chain transfer (RAFT) polymerization, as described in the examples that follow. The embodied thermo-responsive block copolymers comprise: 1) a hydrophilic block, which is an alkyl ether terminated polyethylene glycol (polyethylene oxide); and 2) a thermo-responsive block, which comprises an N-alkylacrylamide, N-alkylaminoacrylate or a copolymer thereof.

Like phospholipid amphiphiles, block copolymers self-assemble in aqueous solution into lamellar phases at certain compositions and temperatures and can form closed bilayer structures capable of encapsulating aqueous materials. Vesicles from block copolymers have the additional advantage of being made from synthetic molecules, permitting one of ordinary skill to apply known synthetic methods to greatly expand the types of vesicles and the material properties that are possible based upon the presently disclosed and exemplified applications. Advantageously, in one embodiment, the thermo-responsive polymersomes comprise a thermo-responsive block copolymer that is capable of polymersome formation at or near physiological temperature. One such block copolymer comprises a hydrophilic block comprising polyethylene glycol terminated with an alkyl ether, and a thermo-responsive block comprising a poly N-alkylacrylamide, poly N-alkylaminoacrylate, or copolymer thereof.

The thermo-responsive block PNIPAAm has a low critical solution temperature (LCST) in water of approximately physiological temperature, i.e., ca. about 32° C. (Li et al., Biomacromol. 6(2):994-999 (2005)). It exhibits remarkable hydration and dehydration transitions in a narrow temperature window (˜10° C.). When the LCST is at, or slightly above body temperature at about 37° C., the thermo-responsive block displays hydrophobic properties, such that the block copolymer self-assembles to form a polymersome with the thermo-responsive block occupying the core of the polymersome and the hydrophilic block occupying the corona of the polymersome. This permits encapsulation of the active agent. Below the LCST, the thermo-responsive block displays hydrophilic properties, such that the polymersome dissociates, thus releasing the encapsulated active agent in a controlled manner. Thus, the morphology of the polymersome that is formed according to the current invention is a function of the weight fraction of the hydrophilic block in the block copolymer and the number average molecular weight (Mₙ) of the block copolymer.

Vesicles can be prepared by any method known to one of ordinary skill in the art. Although the RAFT method is exemplified, the diblock copolymers used to form the vesicles of the invention may also be synthesized by any method known to one of ordinary skill in the art for synthesizing narrow dispersed block copolymers. Such methods, including anionic polymerization [Hillmyer et al., Macromol. 29, 6994 (1996); and Hillmyer et al., Science 271:976 (1996))], atom transfer radical polymerization (ATRP) (Matyjaszewski et al. J. Am. Chem. Soc. 119:674-680 (1997)), nitroxide mediated polymerization (NMP) (Dao et al. J. Polym. Sci. A: Polym. Chem., 36:2161 (1998); Benoit et al. J. Am. Chem. Soc. 121:3904 (1999)), group transfer polymerization (GTP) (Webster and Sogah, In Comprehensive Polymers Science, Vol 4, Eastmond, Ledwith, Russo, Sigwalt, Eds., Pergamon Press, London, 1989, pp 163-169), and RAFT (Journal of Polymer Science: Part A: Polymer Chemistry Vol. 44, 5809-5831 (2006)), although the practitioner need not be so limited. Use of the Bates method results in very low polydispersity indices for the synthesized polymer (not exceeding 1.2), making such methods particularly suited for use in the present invention, at least from the standpoint of homogeneity. Indeed, the demonstrated ability to make stable vesicles from PEO-PEE with up to at least 20% mole fraction of phospholipid strongly indicates that polydispersity need not be limiting in the formation of stable vesicles.

### B. Characterization of Polymersomes

The strong temperature dependence of the block copolymer assemblies provides a new mechanism for cold-controlled delivery and release of an active agent, such as drugs, other therapeutic compositions, proteins, dyes, etc. The structure of an exemplified polymersome vesicle can be characterized by the following generalized method. PEO-b-PNIPAm assemblies in water, 5 mg mL-1 OPA215 was dissolved in water and incubated at 37° C. overnight to promote the micelle formation. A hydrophobic fluorescence dye (PKH26) was then added to the solution to label the assemblies for direct visualization under the fluorescent microscope. For fluorescence imaging, a 2 ml solution was transferred to a Petri dish, which is fixed on a temperature controller, and imaged by, e.g., an Olympus IX71 inverted fluorescence microscope using a 1 OOX objective and a Cascade CCD camera.

Small angle X-ray and neutron scattering (SAXS and SANS) analyses are well suited for quantifying the thickness of the membrane core (Won et al., Science 283:960-3 (1999)) or any internal structure. SAXS and SANS can provide precise characterization of the membrane dimensions, including the conformational characteristics of the PEO corona that stabilizes the polymersome in an aqueous solution. Neutron contrast is created by dispersing the vesicles in mixtures of H₂O and D₂O, thereby exposing the concentration of water as a function of distance from the hydrophobic core.

Size distribution can be determined directly by microscopic observation (light and/or electron microscopy), by dynamic light scattering, or by other known methods. Polymersome vesicles can range in size from tens of nanometers to hundreds of microns in diameter. According to accepted terminology developed for lipid vesicles, small vesicles can be as small as about 1 nm in diameter to over 100 nm in diameter, although they typically have diameters in the tens of nanometers. Large vesicles range from 100 to 500 nm in diameter. Both small and large vesicles are best perceived as such by light scattering and electron microscopy. Giant vesicles are generally greater than 0.5 to 1 μm in diameter, and can generally be perceived as vesicles by optical microscopy.

Small vesicles can be as small as 1 nm in diameter to over 100 nm in diameter, although they typically have diameters in the tens of nanometers. Large vesicles range from 100 to 1000 nm in diameter, preferably from 500 to 1000 nm. Giant vesicles are generally greater than 1 μm in diameter. The preferred polymersome vesicles range of 20 nm to 100 μm, preferably from 1 μm to 75 μm, and more preferably from 1 μm to 50 μm.

The disclosed methods of preparation of the polymersomes are particularly preferred because the vesicles are prepared without the use of co-solvent. Any organic solvent used during the disclosed synthesis or film fabrication method has been completely removed before the actual vesicle formation. Therefore, the polymersomes of the present invention are free of organic solvents, distinguishing the vesicles from those of the prior art and making them uniquely suited for bio-applications. Thermo-responsive polymersomes have been shown to be stable to physiological buffers for more than 10 hours, and they remain quite stable based on drug encapsulation studies. Thus, the vesicles can be used in applications that require long-term storage of material, and crosslinking is not necessary.

Monitoring temperature-dependent polymer assemblies in water by dynamic light scattering (DLS) and UV-vis Spectrometer. OPA215 was dissolved in water at room temperature at a concentration of 0.25 mg ml⁻¹ and filtered into cuvettes directly through 0.45 μm Nylon filters (Whatman) for DLS and UV-vis measurement, respectively. The DLS measurements were carried out on a protein solution DynaPro instrument. The scattering angle was fixed at 90 degrees. The temperature of the solution was controlled within +0.1° C., and the data was analyzed using Dynamics Version 5.26.37. Transmittance at 500 nm was monitored by a Cary 5000 UV-vis-NIR spectrophotometer (Varian Scientific Instrument) fitted with a digital temperature controller (reproducibility of ±0.03° C.).

The methods of analysis applied in a preferred embodiment of the invention provide a clear basis for applications of mass retention, delivery, and extraction, which may require membrane biocompatibility, and which takes advantage of the novel thermal properties of the membranes. By “biocompatible” is meant a substance or composition which can be introduced into an animal, particularly into a human, without significant adverse effect. For example, when a material, substance or composition of matter is brought into a contact with a viable white blood cell, if the material, substance or composition of matter is toxic, reactive or biologically incompatible, the cells will perceive the material as foreign, harmful or immunogenic, causing activation of the immune response, and resulting in immediate, visible morphological changes in the cell. A “significant” adverse effect would be one which is considered sufficiently deleterious as to preclude introducing a substance into the patient.

To confirm biocompatibility of the polymersomes, preliminary evaluations may be performed by bringing the polymersomes into contact with white blood cells, such as granulocytes. If there are adhesions between vesicles and blood cells, micropipette aspiration could be used to measure the inter-lamellar adhesion energy. If two vesicles or a cell and vesicle are manipulated into contact and adherent, then the inter-lamellar adhesion energy density γ is determined from Young's equation, γ=τ(1-cos θ), where θ is the measurable contact angle between the two surfaces, τ is the tension required to peel the membranes apart. In the case of adhesion being strong enough to induce membrane cohesion, aspiration can again be used to directly observe the resulting coalescence of two vesicles (fusion), as well as the adsorption and intercalation of soluble objects (such as, surfactants or micelles) into the membrane.

C. Encapsulation into Polymersomes

A wide range of materials can be encapsulated within a thermo-responsive polymersome vesicle. In fact, to date no molecule has been found that cannot be encapsulated. Among the exemplary active agent molecules that have been encapsulated are: proteins and proteinaceous compositions, e.g., myoglobin, hemoglobin and albumin, sugars and other representative carriers for drugs, therapeutics and other biomaterials, e.g., 10 kDa dextran, sucrose, and phosphate buffered saline, as well as marker preparations. Encapsulation applications range, without limitation from, e.g., drug delivery (aqueously soluble drugs), to optical detectors (fluorescent dyes), to the storage of oxygen (hemoglobin).

A variety of fluorescent dyes of the type that can be incorporated within the polymersomes could include small molecular weight fluorophores, such as FITC, and fluorophores attached to dextrans of a laddered sequence of molecular weights, see, U.S. Patent Applications US2005/0003016, US2005/0048110, US2005/0180922, and U.S. Pat. No. 6,835,394, supra. Imaging of the fluorescent core can be accomplished by standard fluorescent videomicroscopy. Permeability of the polymersome to the fluorophore can be measured by manipulating the fluorescently-filled vesicle with aspiration, and monitoring the retention of fluorescence against a measure of time.

The encapsulation of globular proteins by film rehydration has been demonstrated by U.S. Patent Applications US2005/0003016, US2005/0048110, US2005/0180922, and U.S. Pat. No. 6,835,394, supra.

It is clear from the foregoing, that polymersomes are particularly useful for the transport (either delivery to the bulk or removal from the bulk) of active agents, e.g., hormones, proteins, peptides or polypeptides, sugars or other nutrients, drugs, medicaments or therapeutics, including genetic therapeutics, steroids, vitamins, minerals, salts or electrolytes, genes, gene fragments or products of genetic engineering, dyes, adjuvants, biosealants and the like, but the key to their effectiveness is combining the block copolymers in a manner that provides a method for controlling the release of the encapsulated active agent at a time and location where the released composition is most useful. In fact, the thermo-responsive, stable vesicle morphology of the polymersome is particularly suited to the delivery of biosealants to a wound site. In bioremediation, the polymersomes could effectively transport waste products, heavy metals and the like. In electronics, optics or photography, the polymersomes could transport chemicals or dyes. Moreover, these stable polymersomes may find unlimited mechanical applications, including insulation, electronics and engineering.

In addition, the thermo-responsive polymersome vesicles are ideal for intravital drug delivery because they are biocompatible; that is they contain no organic solvent residue and are made of nontoxic materials that are compatible with biological cells and tissues. Thus, because they can interact with plant or animal tissues without deleterious immunological effects, any drug deliverable to a patient could be incorporated into a biocompatible polymersome for delivery. Adjustments of molecular weight, composition and polymerization of the polymer can be readily adapted to the size and viscosity of the selected drug by one of ordinary skill in the art using standard techniques.

Additional encapsulation applications that involve incorporation of hydrophobic molecules in the bilayer core, but which do not require biocompatibility include, e.g., alkyd paints and biocides (e.g., fungicides or pesticides), obviating the need for organic solvents that may be toxic or flammable. Polymersomes also provide a controlled microenvironment for catalysis or for the segregation of non-compatible materials.

The thermo-responsive vesicles of the present invention further provide useful tools for the study of the physics of lamellar phases. At different temperatures or reduced volumes (achieved by deflating the vesicle interior with an external high salt solution), such vesicles will display a variety of shapes. Comparison between observed shapes and theoretical calculations are used to verify theoretical concepts of how lamellar phases behave, e.g., features, such as the curvature, or the tendency of molecules to “flip-flop” between monolayers.

The present invention is further described in the following examples. These examples are not to be construed as limiting the scope of the appended claims.

### EXAMPLES

### Example 1

### Temperature Controlled Assembly and Release from Polymersomes

To demonstrate the feasibility of the reversible addition-fragmentation chain transfer (RAFT) polymerization technique (Scheme 1) (see Hong et al., J. Polym. Sci. Pol. Chem. 42:4873 (2004)), a series of well-defined PEO-b-poly-(N-propylacrylamide) (PEO-PNIPAAm) block copolymers (ƒ_{EO}, 7.6-66.6 wt %) were synthesized with narrow distribution (PDI<1.2). PEO-PNIPAAm, according to the present invention was produced from poly (ethylene glycol) methyl ester (PEO) having number average molecular weights (Me) of 2000 and 5000, respectively, using reversible addition-fragmentation chain transfer (RAFT) polymerization. The poly(ethylene glycol) methyl ester was obtained from Sigma-Aldrich Milwaukee, Wis. The poly(ethylene glycol) was terminated with a methyl ester at one end and had a free hydroxyl at the opposite end.

Materials. Dithiobenzoic acid (DTBA) was synthesized according Thang et al., Tetrahedron Lett. 40:2435 (1999). Purity was confirmed by ¹H NMR spectrum. Poly(ethylene glycol) methyl ester (from Aldrich with Mₙ=2000) and maleic anhydride (from Aldrich, 99%) were used as received. N-isopropylacryl-amide (from Aldrich, 97%) was recrystallized from hexane. Other reagents and solvents were used as received from Aldrich or Acros Chemicals.

Synthesis of the Macrotransfer Agent for RAFT. A 250 ml round bottom flask was charged with 20.0 g (10.0 mmol) of PEO (Mₙ=2000), 10.0 g (102.0 mmol) of maleic anhydride and 200 ml of anhydrous toluene. The mixture was stirred at 65° C. for 24 hours. The toluene was removed by evaporation and the residue was dissolved in 150 ml of methylene chloride. PEO with one maleic acid terminal group was purified by repeated precipitation from methylene chloride into petroleum ether until the unreacted maleic anhydride was removed as evidenced by proton NMR.

Synthesis of dithiobenzoyl end group functionalized PEO (PEO-DTB). A 100 ml round bottom flask was charged with 12.0 grams (5.7 mmol) of the mono-maleic anhydride (MAA; Mₙ=2000) terminated PEO, 9.1 grams (57.0 mmol) dithiobenzoic acid (DTBA) and 70 ml of tetrachlorocarbon (carbon tetrachloride). The mixture was stirred at 70° C. for 20 hours. The resulting dithiobenzoic acid functionalized PEO (PEO-DTB) was purified by repeated precipitation from methylene chloride into petroleum ether until the unreacted DTBA was removed as evidenced by proton NMR. After drying in a vacuum oven overnight at room temperature, the PEO-DTB was refrigerated.

The general procedure discussed above is illustrated in Scheme 1.

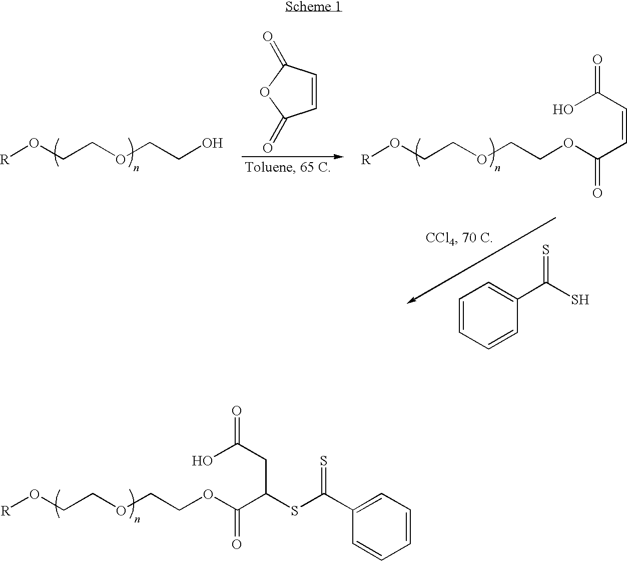

Preparation of Peo-b-PNIPAAm by Raft Polymerization. The general synthesis followed for production of PEO-b-PNIPAAm was as follows: A 50 ml dried Schlenk flask was charged with 0.45 g (0.2 mmol) of dithiobenzoic acid functionalized PEO, 2.0 grams (17.7 mmol) of N-isopropylacrylamide, 2 mg (0.012 mmol) of 2,2′-azo-bis-isobutyronitrile (AIBN) and 35 ml of dioxane. After degassing through three freeze-pump-thaw cycles, the flask was placed in a thermostated oil bath at 100° C. for a set time. The resultant PEO-b-PNIPAAm was purified by precipitation into diethyl ether or petroleum ether.

Characterization of PEO-b-PNIPAAm copolymers. Molecular weights of block copolymers were measured using gel permeation chromatography (GPC) on a GPC50 (Polymer Labs, now Varian, Inc, Palo Alto, Calif.) and ¹H NMR spectra (frequency), respectively. The PL-GPC50 system is equipped with a PL MIDAS 830 autosampler, PLgel 5μ MIXED-C columns, and a PL COM9 RI detector against linear polystyrene standards in THF (1 ml/min) at 27° C. ¹H NMR spectra of the polymers were obtained on a Bruker DMX-300 Hz spectrometer.

Preparation of Peo-b-PNIPAAm self-assemblies and Visualization. Peo-b-PNIPAAm was dissolved in water (5 mg ml⁻¹) and incubated at 37° C. overnight to promote the micelle formation. A hydrophobic fluorescence dye (PKH26) was then added to the solution to label the assemblies for direct visualization under the fluorescence microscope. A 2 ml solution was transferred to a Petri dish, which is fixed on a temperature controller, and imaged by Olympus IX71 inverted fluorescence microscope using a 100× objective and a Cascade CCD camera. The morphologies of block copolymer assemblies were imaged at different temperatures ˜32+10° C.

Synthesis. As shown in Scheme 1, monofunctional poly(ethelene glycol) methyl ester (PEO-OH) reacted with maleic anhydride to introduce a double bond through esterification, followed by addition reaction of dithiobenzoic acid (DTBA) to the double bond in tetrachlorocarbon. The reaction of PEO-OH with maleic anhydride yields maleic acid terminated PEO (PEO-MAA). The complete functionalization of the hydroxyl end group was confirmed by 1H MNR spectrum, in which the integration ratio of peaks at 6.4, 6.2 and 3.4 ppm, corresponding to protons of double bonds and the methyl end of PEO, respectively is 1:1:3. The disappearance of peaks at 6.4 and 6.2 ppm, and the appearance of aromatic protons at 7.0-8.0 ppm, whose ratio to that of CH₃ from the PEO end is 5:3, indicating complete conversion of the double bond to the dithiobenzoate.

The RAFT polymerization synthetic scheme was applied to a series of well-defined PEO-b-PNIPAAm block copolymers to study their self-assembly behaviors in aqueous solution at various temperatures.

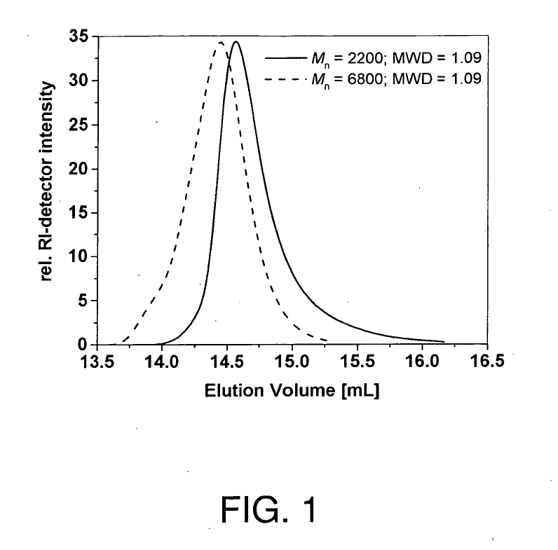

As shown in FIG. 1, the formation and dissociation of vesicles in aqueous solution visualized by fluorescence microscopy, both traces of the macromolecular agent PEO-DTB (solid line), and its corresponding block copolymer, PEO-b-PNIPAAm (dashed line) (Mₙ of 25000, i.e., a hydrophilic fraction of about 8%), produced from poly(ethylene glycol) methyl ether, (Mₙ=2000), have symmetrical unimodal peaks. However, the temperature dependence of self-assembly and dissociation of the temperature-responsive block copolymer is seen in FIG. 1 as the block copolymer peak moves significantly towards higher molecular weight, clearly supporting the formation of well-defined block copolymer with narrow molecular weight distributions, i.e., MWD<1.20.

Morphology of polymersomes Formed by PEO-b-PNIPAAm Copolymer Assemblies. PEO-b-PNIPAAm possesses a lower critical solution temperature (LCST) in water of 32° C., and exhibits remarkable hydration and dehydration transitions in a narrow temperature window (˜10° C.). At a temperature below the LSCT, intermolecular hydrogen bonding is formed between water and PINAAM, resulting in hydration and swelling of the polymer chains. However, when temperature increases, the polymer chains collapse out of water and intramolecular hydrogen bonding is dominant, with hydrophobic isopropyl groups exposed to the water, demonstrating the present method for manipulating the self-assembly of the PEO-b-PNIPAAm in water for controlled encapsulation and release of an active agent in response to temperature.

Spherical, cylindrical micelles (including branched worms), and vesicles are the three stable morphologies, whose formation depends on the average molecular weight (Mₙ) of the block copolymer and the weight fraction of each block (the hydrophilic PEO and the thermo-responsive fractions of the copolymer) in the amphiphile. Previous studies demonstrated a relationship between the self-assembly of PEO-b-PLA in aqueous solution: for vesicles, ƒEO=0.20-0.40; for cylindrical micelles, ƒEO=0.42-0.50; and for spherical micelles, ƒEO=>0.50. For PEO-b-PNIPAAm, below the LCST, PNIPAAm blocks become hydrophilic and water is a good solvent for both PEO and PNIPAAm blocks. The block copolymer adopts a randomly coiled conformation. Above the LCST, the PNIPAAm becomes hydrophobic and collapses into a globular conformation. Therefore, the double hydrophilic block copolymer PEO-b-PNIPAAm becomes amphiphilic in aqueous solution when the temperature increases above the LCST of PNIPAAms.

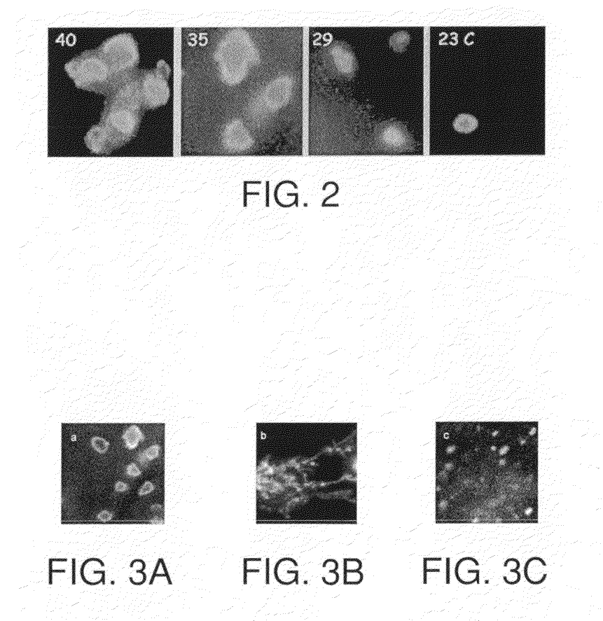

The morphology of PEO-b-PNIPAAm copolymer in aqueous solution was further characterized by fluorescence using the hydrophobic fluorescent dye, PKH26, representative of the active agent. Below the LCST the dye dispersed in the whole solution because of the hydrophilic property of the solution. Above the LCST, the hydrophobic dye was adsorbed in the hydrophobic PNIPAAm segments only, and imaged the morphology of the copolymer assemblies. FIG. 2 shows the typical temperature-dependent assembly in aqueous solution. The vesicles formed by a PEO-b-PNIPAAm block copolymer having a Mₙ of 25000 and a PEO fraction of 8%. Above the LCST for the copolymers, the block copolymer self-assembled as highly ordered vesicles at 35° C. (>LCST), and the vesicles became more crowded at 40° C. When the temperature was decreased to 29° C. (<LCST) the vesicles dissociated to very small vesicles or disappeared altogether. At 23° C. almost all of the vesicles had disappeared, which resulted in the hydrophobic dye dissociating and dispersing into the aqueous solution again.

FIG. 3 shows the highly ordered vesicles, branched worms and short rods formed by the PEO-b-PNIPAAm block copolymer depend on the molecular weight and weight fraction of the two block segments in aqueous solution above the LCST of PNIPAAm. The highly ordered vesicles have Mₙ of 25,000; and ƒ_{EO} of 8%; the branched worms have a M, of 5,500; and ƒ_{EO} of 36%; short rods have a Mₙ of 3,500; and ƒ_{EO} of 58%. The two block segments have similar temperature responsiveness.

Consequently, well-defined thermo-responsive block copolymers, PEO-b-PNIPAAm have been synthesized from two PEO macroinitiators capped with a dithiobenzoyl group, Mₙ, _{EO}=2000 and 5000 g/mol, respectively, by RAFT polymerization. When the block copolymers self-assemble into different morphologies, depending on the molecular weight and weight fraction of each of the two block segments, the self-assembly was shown to be temperature sensitive.

### Example 2

### Properties of a Polymer Vesicle of PNIPAAm

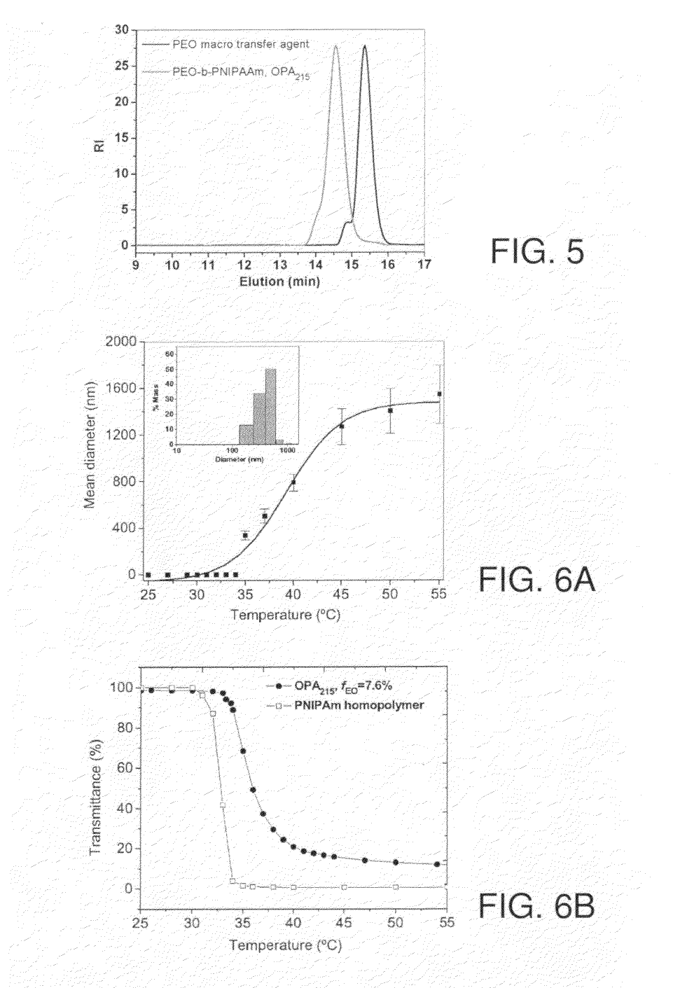

To further examine the properties of the thermo-responsive polymersome of Example 1, since morphology of soft assemblies of amphiphilic block copolymers in water was shown to be determined by the weight fraction of the hydrophilic block (ƒₚₕᵢₗ), the molecular weight of the polymer (N), and the effective interaction parameter of the core block with H₂O (χ), the following experiment focused on the temperature-dependent assembly/disassembly behavior of vesicle structures made from block copolymers with the PEO fraction (ƒ_{PEO})=7.6 wt %. For block copolymers with a large χ, vesicles are favored when ƒ_{PEO} is in the range of 20-40 wt %. To determine whether copolymerization of PEO with a temperature-responsive block, such as PNIPAAm, copolymer OPA₂₁₅, was synthesized, where the subscript represents the degree of polymerization of the NIPAAm block. The PDI of OPA₂₁₅ is 1.09, based on gel permeation chromatography (GPC) measurements (FIG. 5).

Materials were from Sigma-Aldrich as in Example 1, and the RAFT polymerization method was as indicated in Example 1. The maleic anhydride end-functionalized PEO (PEO-MAA) and dithiobenzoyl end-group functionalized PEO (PEO-DTB) were prepared as in Example 1. The purity of the products was confirmed by ¹HNMR spectra. For a typical synthesis, a 50 ml dried Schlenk flask was charged with 0.45 g (0.2 mmol) PEO-DTB, 2.0 g (17.7 mmol) NIPAm, 2 mg (0.012 mmol) AIBN, and 35 ml 1,4-dioxane. After degassing 3×by freeze-thaw cycles, the flask was sealed and placed in a temperature-controlled oil bath at 100° C. for a preset time. PEO-b-PNIPAm was purified by precipitation into diethyl ether or petroleum ether.

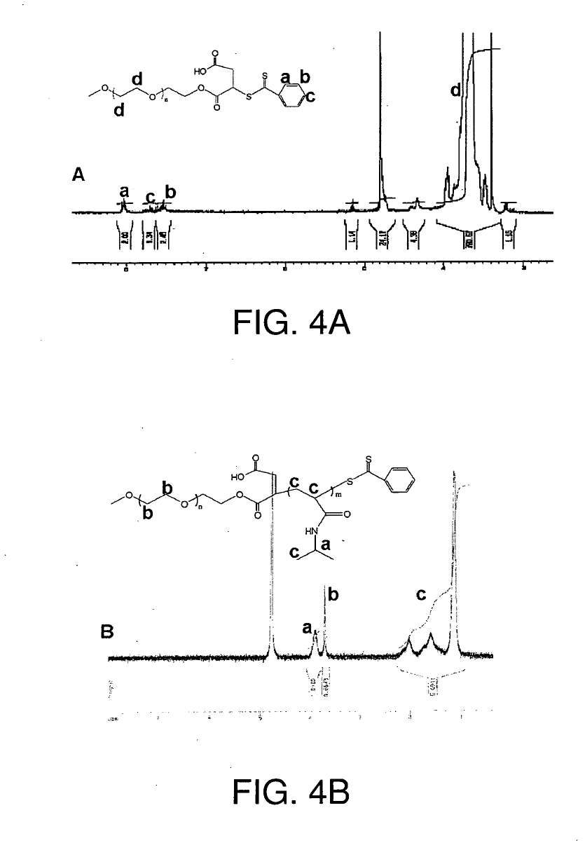

Molecular weights of block copolymers were determined by ¹HNMR (Table 1 and FIG. 4) using a Bruker DMX-300 MHz spectrometer, and by GPC (FIG. 5) using a PL-GPC50 as in Example 1.

TABLE S1
Properties of PEO-b-PNIPAm block
copolymers prepared by RAFT

| Experimental | | | M_{w}/Mₙ | Morphology of |
|---|---|---|---|---|
| entry | f_{PEO} (wt %)^{a} | Mₙ (NMR) | (GPC) | aggregates^{b} |
| 1 | 7.6 | 26,300 | 1.09 | vesicles ~37° C. |
| 2 | 15.3 | 13,100 | 1.11 | vesicles >50° C. |
| 3 | 21 | 11,500 | 1.08 | vesicles >70° C. |
| 4 | 27.5 | 7,300 | 1.12 | clear >90° C. |
| 5 | 32 | 6,250 | 1.17 | N/A |
| 6 | 39 | 5,100 | 1.16 | N/A |
| 7 | 44.3 | 4,500 | 1.18 | N/A |
| 8 | 74.1 | 2,700 | 1.22 | N/A |

^{a}calculated from ¹HNMR spectra;
^{b}observed under fluorescence microscope. Since the objective lens was not heated, the observed transition temperatures might be higher than the actual ones.

At 37° C., large vesicles of OPA₂₁₅ are clearly seen with fluorescence microscopy (FIG. 6). The characteristic membrane structure was labeled with hydrophobic dye (PKH 26) molecules as above. When the temperature was decreased to 25° C., the vesicles quickly dissociated, releasing the integrated dyes and causing the vesicle fluorescence to disappear. Although the fluorescence images provide convincing proof of the formation of thermo-responsive vesicles up to a few micrometers in diameter, the density of these large vesicles was typically low, and many smaller vesicles (and perhaps spherical micelles) appeared to be diffusing in the background solution.

The low resolution of fluorescence microscopy and the rapid motion of small objects limits direct imaging of aggregates smaller than ca. 500 nm in diameter. Therefore, dynamic light scattering (DLS) measurements were performed to confirm the temperature-driven assembly and disassembly of OPA₂₁₅ vesicles. DLS is well suited to quantitatively analyze the size distributions of polymer aggregates smaller than ca. 1 μm, and thus, provides information complementary to fluorescence microscopy. The mean hydrodynamic diameter, Dₕ, of the polymer micelles was followed while raising the solution temperature from 25° to 55° C. (FIG. 6A). No aggregates were detected below 33° C., indicating that the block copolymers were individual chains dispersed in solution. As the temperature approached 33° C., aggregates with Dₕ>300 nm began to form, quickly increasing in size to above 1 μm at temperatures above 40° C. Compared to the PNIPAAm homopolymers, which exhibit an LCST of ca. 32° C. (Wu et al., Phys. Rev. Lett. 80:4092 (1998)), OPA₂₁₅ has a slightly higher LCST (ca. 36° C.) and a broader transition, as determined from both DLS and turbidity measurements. Thus, while large error bars are intrinsic to DLS when the Dₕ value is greater than 1 μm, vesicle formation from OPA₂₁₅ above the LCST was clear. A Dₕ>>100 nm (see inset in FIG. 6A) is larger than the much smaller diameters of spherical micelles (<50 nm) seen with strongly segregated block copolymers of a similar molecular weight, and also as compared to the 78-227 nm range formed with narrowly dispersed PEO-b-PNIPAAm (Zhang et al, supra, 2005).

The phase transition of the block-copolymer assemblies was further investigated by monitoring optical transmission at 500 nm (FIG. 6B). Consistent with the DLS results, the polymer solution proved transparent below 33° C., and the transmittance dropped quickly when the temperature was increased as the polymersomes formed.

### Example 3

### Fluidicity and Robustness of Polymersome Membrane

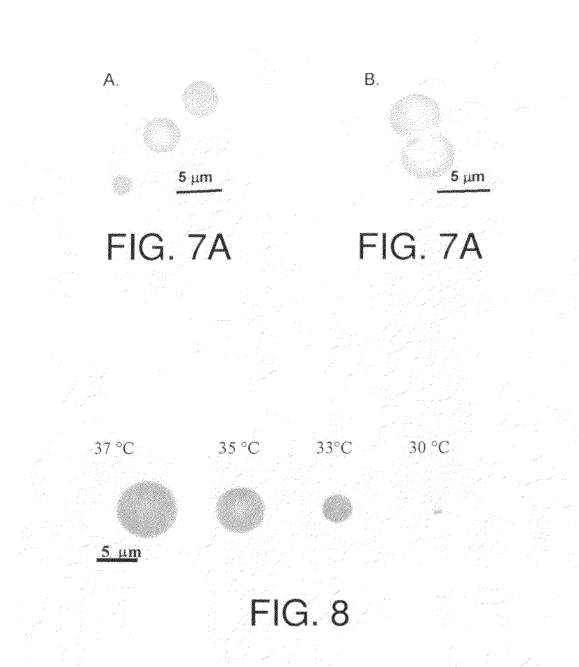

Because it is important to maintain the fluidicity (as in a liposome) while increasing the membrane stability using polymersomes, the fluidicity and robustness of the polymer vesicle membrane was studied. Hydrophilic sucrose was encapsulated into thermo-responsive polymersomes formed by the RAFT method above at different osmotic pressures, and the vesicle structure was monitored using bright field phase contrast microscopy. A 5 mg ml⁻¹ of OPA₂₁₅ aqueous solution containing 340 g mol⁻¹ sucrose (320 mOsm) was incubated at 37° C. overnight, followed by centrifugation at 40° C. The separated sucrose encapsulating vesicles were then suspended into a 320 mOsm isotonic PBS solution. Since the refractive index of the encapsulated sucrose and the external isotonic solution of PBS are rather different, sucrose appears dark inside the vesicles. As seen in FIG. 7A, the vesicles appeared smooth, spherical and phase dark in PBS solution at 37° C., indicating that the sucrose was retained inside the vesicles.

When suspending into a 400 mOsm PBS solution, the spherical vesicle was deflated into a double-bell shape (FIG. 7B) because of the increased external osmotic pressure. However, the vesicle's contour maintained smooth, suggesting that the vesicle membrane was fluidic, yet robust enough to protect the encapsulated molecules. The PNIPAAM chains may rearrange themselves significantly within the surface to relax accumulated strain, i.e., the membranes of the vesicles are fluidic such that they can adjust geometry to equilibrate the osmotic pressure difference between the inside and outside of vesicles.

Next, cold-controlled release was performed of the encapsulated sucrose from vesicles in an isotonic PBS solution. As shown in FIG. 8, the size of sucrose encapsulated vesicle decreased when lowering temperature, and sucrose was released nearly completely at 30° C., which agreed well with the observed temperature-dependent disassembly of polymer vesicles. It is worth noting that during the disassembly process the sucrose-encapsulated block copolymer vesicle remained smooth and spherical, consistent with the previous conclusion that the vesicle membrane is fluidic and robust.

The fact that vesicles self-assembled from OPA₂₁₅ can encapsulate and release molecules triggered by a small temperature change (˜10° C.) near the physiological temperature is important for applications such as temperature-responsive drug delivery.

### Example 4

### Thermo-Responsive Polymersome as a Drug Delivery Vehicle

Because vesicles that self-assembled from OPA₂₁₅ appeared capable of encapsulation and release of molecules when triggered by a small decrease in temperature (below body temperature, 37° C.), temperature-controlled drug release from OPA₂₁₅ vesicles was examined. Doxorubicin (Dox) is an anticancer drug that is both water-soluble and membrane-permeable at neutral pH. It is widely used in the treatment of solid tumors and leukemia. However, the cardiotoxicity of the free drug limits dosage. Liposomes have been developed as Dox delivery carriers that can reduce the accumulation of the drug in the heart, and as a result polymersome encapsulation of Dox was expected to reduce leakage and also to provide novel release mechanisms compared to liposomes.

To prepare the PEO-b-PNIPAAm assemblies in water, 5 mg ml⁻¹ OPA₂₁₅ was dissolved in water and incubated at 37° C. overnight to promote micelle formation. PKH 26 was added to the solution to label the assemblies for direct visualization by fluorescence microscopy. A 2 ml solution was transferred to a Petri dish fixed on a temperature controller, and imaged by inverted fluorescence microscopy as in Example 1.

To monitor the temperature-responsive polymer assemblies in water by DLS and UV-vis spectrometry, OPA₂₁₅ was dissolved in water at room temperature at a concentration of 0.25 mg ml⁻¹ and filtered into cuvettes directly through 0.45 μm nylon filters (Whatman). DLS measurements were carried out on a protein solution DynaPro instrument. The scattering angle was fixed at 90°. The temperature of the solution was controlled within ±0.1° C., and data was analyzed using Dynamics version 5.26.37. Transmittance at 500 nm was monitored by a Cary 5000 UV-vis-NIR spectrophotometer (Varian Scientific Instruments) fitted with a digital temperature controller (reproducibility of ±0.03° C.).

Encapsulation and Release of Dox within PEO-b-PNIPAm Vesicles. OPA₂₁₅ (10 mg ml⁻¹) and Dox (200 μg ml⁻¹) were mixed and Dox was entrapped in the OPA₂₁₅ vesicles (by standard methods of encapsulation), with an acid gradient across the membrane (incubation in 0.320 osM citrate buffer solution (pH 4) at 37° C. overnight). Since Dox in its HCl salt form has a higher solubility in citric acid than in phosphate-buffered saline (PBS), Dox was first dissolved in citric acid, followed by an increase in temperature to 37° C. The Dox-loaded vesicles were centrifuged at 40° C. to remove excess free Dox and resuspended into a 0.320 osM PBS solution at ° C. Then, 3 ml of the aforementioned solution was injected into a dialysis cassette (molecular weight cut-off 3000 g mol⁻¹), and dialyzed against the PBS solution at 37° C. for 6 hours to ensure complete removal of any free Dox outside of the vesicles. After the initial aliquot was taken, the cassette was immersed in a 300 ml PBS solution at 37° and 27° C., respectively, for monitoring the 485 nm absorption peak of Dox using UV-vis spectrometry. At time intervals, a 3 ml aliquot of the PBS solution was sampled from the external solution of the dialysis cassette.

The Dox is most likely incorporated into the vesicle lumen because Dox is water soluble and positively charged when interacting with citric acid inside the vesicle. However, interactions of Dox with PNIPAAm in the vesicle membrane are also possible. Nevertheless, regardless of the mechanism, these studies indicate that cancer drugs (e.g., doxorubicin) encapsulated in the vesicles have long-term stability at 37° C. in contrast to the free drugs, and the active agents are quickly released at room temperature (“cold-controlled” release).

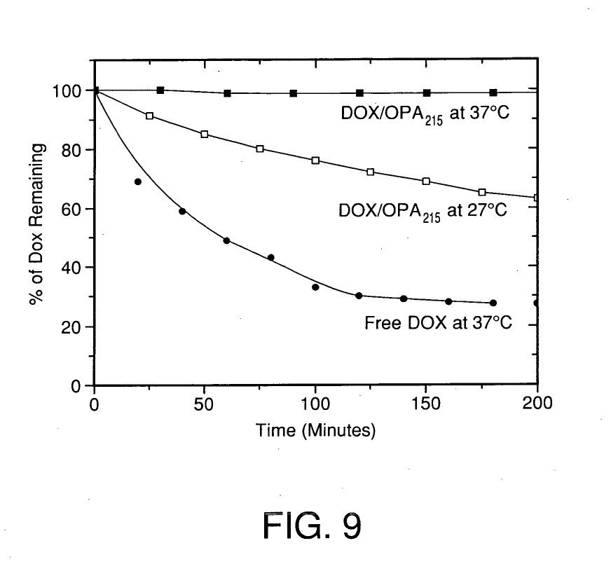

To study thermally triggered release of Dox from the OPA₂₁₅ vesicles in PBS solutions, vesicles and control samples were entrapped in dialysis cassettes and sampled in external PBS solutions for the 485 nm absorption peak of Dox at different time intervals (FIG. 9). Clearance of free Dox during dialysis at 37° C. was used as reference, which occurred within 2 hours (>70% loss). In contrast, vesicle-encapsulated Dox proved to be far more stable at 37° C., i.e., no detectable amount of Dox was released from the vesicles for up to 7 hours. When cooled to 27° C., the PNIPAAm cores within the vesicles shifted from hydrophobic to hydrophilic, inducing pores in the vesicles or causing general rupture, triggering release of Dox. The initial rate of release was 18% hour⁻¹. Since about half of the Dox remained in the dialysis bag at 27° C. after 200 min, it seems likely that interactions between uncharged Dox and the hydrophilic PNIPAAm chains at room temperature foster such retention. Nonetheless, the PEO-b-PNIPAm copolymer vesicles have displayed positive results for use for encapsulating and delivering drugs by thermo-responsive controlled release in vivo when coupled, e.g., to hypothermic patches and local cryosurgery probes.

In summary, well-defined PEO-b-PNIPAAm block copolymers (PDI≦1.2) have been synthesized using RAFT polymerization, and when formed into thermo-responsive polymersomes, they displayed temperature-dependent assembly/disassembly capability. The narrow phase-transition window (ca. 10° C.) makes the vesicles attractive for targeted transport and release. The PEO-b-PNIPAm vesicles are shown to be stable at body temperature and also to encapsulate both hydrophilic drugs (e.g., Dox) and integrate hydrophobic molecules into their membranes (e.g., PKH 26), while allowing temperature-controlled quick release of both types of compounds below 32° C. Accordingly the present invention provides methods utilizing the design and self-assembly of such thermo-responsive block copolymers to provide new and therapeutically useful means for temperature-controlled, site-specific release of various hydrophilic or hydrophobic active agents, ranging from dyes and nutrients to drugs, nucleic acids, proteins and the like. The encapsulated thermo-responsive polymersomes are particularly suited for the delivery of in vivo therapeutics. They can be stored at body temperature or circulate in vivo without any burst effect. Once they reach a targeted site, the encapsulated active agent can be released locally using a cold patch, temperature-controlled probe or cathode.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

What is claimed is:

1. A thermo-responsive polymersome comprising a block copolymer comprising:
hydrophilic block, comprising polyethylene glycol terminated with an alkyl ether, and
thermo-responsive block, comprising a poly N-alkylacrylamide, poly N-alkylaminoacrylate or copolymer thereof, wherein,
above a lower critical solution temperature the thermo-responsive block displays hydrophobic properties, such that the block copolymer self-assembles to form a polymersome with the thermo-responsive block occupying a core of the polymersome and the hydrophilic block occupying a corona of the polymersome, and
below the lower critical solution temperature the thermo-responsive block displays hydrophilic properties, such that the polymersome dissociates; and
wherein the morphology of the polymersome is a function of the weight fraction of the hydrophilic block and the number average molecular weight of the block copolymer.

2. The polymersome of claim 1, wherein the block copolymer is formed by adding the thermo-responsive block to the hydrophilic block by reversible addition-fragmentation chain transfer (RAFT) polymerization.

3. The polymersome of claim 1, wherein the hydrophilic block has a number average molecular weight of ranging from about 2000 to 5000.

4. The polymersome of claim 3, wherein the block copolymer has a number average molecular weight ranging from 3500 to 25000.

5. The polymersome of claim 1, wherein the block copolymer has a molecular weight distribution of 1.2 or less.

6. The polymersome of claim 1, wherein the lower critical solution temperature is about 32° C.

7. The polymersome of claim 1, wherein the block copolymer self-assembles into highly ordered vesicles.

8. The polymersome of claim 1, wherein the block copolymer self-assembles into branched worm micelles.

9. The polymersome of claim 1, wherein the block copolymer self-assembles into short rod micelles.

10. A method for encapsulating a hydrophilic or hydrophobic active agent in a thermo-responsive polymersome, the method comprising:
providing a block copolymer comprising a hydrophilic block, comprising polyethylene glycol terminated with an alkyl ether, and a thermo-responsive block, comprising a poly N-alkylacrylamide, poly N-alkylaminoacrylate or copolymer thereof, wherein above a lower critical solution temperature the thermo-responsive block displays hydrophobic properties, such that the block copolymer self-assembles to form a polymersome with the thermo-responsive block occupying a core of the polymersome and the hydrophilic block occupying a corona of the polymersome, and below the lower critical solution temperature the thermo-responsive block displays hydrophilic properties, such that the polymersome dissociates; and wherein the morphology of the polymersome is a function of the weight fraction of the hydrophilic block and the number average molecular weight of the block copolymer;
forming an aqueous solution or suspension of the block copolymer and the active agent to be encapsulated;
heating the aqueous solution or suspension to a temperature at or above the lower critical solution temperature, thus triggering self-assembly of the block copolymer into a plurality of polymersomes, thereby encapsulating the active agent therein.

11. The method of claim 10, wherein the hydrophilic block has a number average molecular weight ranging from about 2000 to 5000.

12. The method of claim 11, wherein the block copolymer has a number average molecular weight ranging from about 3500 to 25000.

13. The method of claim 10, wherein the block copolymer has a molecular weight distribution of 1.2 or less.

14. The method of claim 10, wherein the lower critical solution temperature is about 32° C.

15. The method of claim 10, wherein the active agent is selected from the group consisting of therapeutic compound, dye, indicator, biocide, nutrient, protein or protein fragment, salt, gene or gene fragment, steroid, and gas.

16. The method of claim 10, wherein the active agent comprises an active pharmaceutical or therapeutic agent or drug.

17. The method of claim 10, wherein the block copolymer self-assembles into vesicles, branched worm micelles or short rod micelles.

18. A method for thermo-controlled delivery of a hydrophilic or hydrophobic active agent, the method comprising:
providing a block copolymer comprising a hydrophilic block, comprising polyethylene glycol terminated with an alkyl ether, and a thermo-responsive block, comprising a poly N-alkylacrylamide, poly N-alkylaminoacrylate or copolymer thereof, wherein above a lower critical solution temperature the thermo-responsive block displays hydrophobic properties, such that the block copolymer self-assembles to form a polymersome with the thermo-responsive block occupying a core of the polymersome and the hydrophilic block occupying a corona of the polymersome, and below the lower critical solution temperature the thermo-responsive block displays hydrophilic properties, such that the polymersome dissociates;
forming an aqueous solution or suspension of the block copolymer and the active agent to be delivered;
heating the aqueous solution or suspension to a temperature at or above the lower critical solution temperature, thus triggering self-assembly of the block copolymer into a plurality of polymersomes, thereby encapsulating the active agent;
delivering at least a portion of the polymersomes encapsulating the active agent to a target area; and
locally cooling the target area to a temperature below the lower critical solution temperature to cause dissociation of the polymersome, thereby releasing the active agent in a thermo-controlled manner.

19. The method according to claim 18, wherein the lower critical solution temperature is about 32° C.

20. The method of claim 18, wherein the polymersome is biocompatible.

21. The method of claim 20, wherein the method further comprises introducing the polymersomes encapsulating the active agent into a patient and releasing the encapsulated active agent in a thermo-controlled manner at a target site in the patient.

22. The method of claim 20, wherein the active agent is selected from the group consisting of therapeutic compound, dye, indicator, biocide, nutrient, protein or protein fragment, salt, gene or gene fragment, steroid, and gas.

23. The method of claim 22, wherein the active agent comprises an active pharmaceutical or therapeutic agent or drug.

24. The method of claim 18, wherein the block copolymer self-assembles into vesicles, branched worm micelles or short rod micelles.
